(19) United States
(12) Patent Application Publication
(10) Pub. No.: US 2009/0186095 A1
(43) Pub. Date: Jul. 23, 2009

(54) Human hair root stimulator using emu oil to deliver specific therapeutic grade essential oils to the hair follicle

(76) Inventor: Jennifer Lynn Bowers Bahney, Circleville, OH (US)

Correspondence Address: JENNIFER L. BOWERS BAHNEY, 574 WOODLAND DR., CIRCLEVILLE, OH, 43113, US

(21) Appl. No.: 12/009,155

(22) Filed: Jan. 17, 2008

(51) Int. Cl. A61K 35/12; A61P 17/00

(52) U.S. Cl. 424/522

(57) ABSTRACT

The invention utilizes the transdermal properties of oil from the Australian native Emu bird (Dromais Novae-Hollandiae) to deliver the specific therapeutic grade essential oil blend of Rosemary, Thyme, Lavender and Cedarwood through the human scalp to the hair root below, thus stimulating hair growth in live hair follicles.

### REFERENCES CITED

### U.S. Patent Documents

| | | |
|---|---|---|
| 5,494,667 | February 1996 | Uchida, et al. |
| 5,662,921 | September 1997 | Fein, et al. |
| 5,744,128 | April 1998 | Holick |
| 6,193,976 | February 2001 | Porras, et al. |
| 6,303,132 | October 2001 | Nelson |
| 6,528,040 | March 2003 | Pearson, et al. |
| 6,733,751 | May 2004 | Farmer |
| 6,946,144 | September 2005 | Jordan |

### OTHER REFERENCES

Cooksley, Valerie Gennari, Aromatherapy: A Lifetime Guide to Healing with Essential Oils. Prentice Hall, Paramus, N.J., 1996.
Grieve, Mrs. M., A Modern Herbal Volumes I & II. New York, 1971.
Hay, Isabelle, et al., “Successful Treatment For Alopecia Areata,” Archives of Dermatology. 1998; 134:1349-1352, 1996.
Holick, Michael, “Study on Skin Enhancement and Hair Growth,” Drug & Cosmetic Industry Magazine, 1996.
Schatz, Sherrie, et. al., Emu Oil: Reexamining a Natural Remedy with Today's Technology. Emu Today & Tomorrow LLC, Nardin, Okla., 1996.

### BACKGROUND OF THE INVENTION

### 1. Field of the Invention

The invention relates to a composition containing emu oil as the transdermal delivery system for essential oils of rosemary, thyme, lavender and cedarwood, having the effect of penetrating the human scalp and stimulating the hair roots below for the purpose of promoting healthy hair growth in living hair follicles.

### DEFINITIONS

Fully-Refined Emu Oil: Fat extracted from the backs of emu birds that is rendered into a stable oil using the following steps:

1. Grinding the fat into small particles

### 2. Heating the fat

3. Adding deodorizing clay and/or water

### 4. Filtering the oil

5. Vacuum distillation to remove odor

The finished oil should be stored in an air-tight, light blocking container to preserve shelf life. (Schatz, p. 25-26).

The refined oil's composition is almost 100% triglyceride lipid, giving it the following qualities: skin penetrating, anti-inflammatory, antibacterial, healing, moisturizing, non-irritating and non-comedogenic. (Schatz p. 37)

### Essential Oil:

“The exclusive product of the extraction of the volatile aromatic principles contained in the substances of which they bear the name.” Essential oils (also called volatile oils) are highly concentrated forms of the plant part in which they were derived and are removed using the following methods: steam distillation, expression, infusion, alcohol extraction, and benzene or hexane extraction. (Cooksley p. 4,7)

Rosemary Essential Oil: Rosmarinus officinalis. Extracted from the plant's leaves using the distillation method. Properties include: antiseptic, astringent and parasitic. Has been used for centuries as a skin toner and remedy for oily hair, dandruff and hair loss. The chief constituents of the oil are borneol, bornyl acetate and other esters, a special camphor, cineol, pinene and camphene. (Grieve p. 681-683)

Thyme Essential Oil: Thymus vulgaris. Extracted from the fresh-flowering herb using the distillation method. Properties include: antiseptic. Used as a powerful antiseptic for both internal and external use. Chief constituents are the phenols Thymol and Carvacrol. (Grieve, Vol. II, p. 808-813)

Lavender Essential Oil: Lavendula augustifolia. Extracted from the flowers and flower-stalks using the distillation method. Properties include: antiseptic, antiviral, carminative and nervine. Used to remedy skin inflammations. The chief constituents of the oil are linalool and its acetic ester, linalyl acetate. Other constituents are cineol, pinene, limonene, geraniol, borneol and some tannin. (Grieve, Vol. II, p. 467-473)

Cedarwood Essential Oil: Cedrus atlantica. Extracted from the wood using distillation. Properties include: antiseptic, astringent, diuretic, expectorant, fungicidal, sedative, insecticide and tonic. Used on skin infections, oily skin, dandruff and hair loss. (Cooksley p. 343)

Transdermal: relating to, being, or supplying a medication in a form for absorption through the skin into the bloodstream <transdermal drug delivery> (Merriam-Webster online).

Carrier oil: Any oil used to dilute volatile essential oils before they are applied topically to human skin. Examples: jojoba oil, mineral oil, grapeseed oil, coconut oil.

### SUMMARY OF THE INVENTION

The invention presented is a method of stimulating human hair follicles in order to promote healthy hair growth. It consists of a two-part formula: (1) fully-refined emu oil with skin-penetrating abilities that delivers (2) a mixture of Rosemary, Thyme, Lavender and Cedarwood Essential Oils beneath the scalp, directly to the hair follicles below. The preparation is massaged into the scalp and left to penetrate for up to 12 hours before being washed out. The mixture may be applied at home by an individual or in a salon by a salon professional.

### DETAILED DESCRIPTION

The invention consists of a topical scalp-penetrating hair growth preparation of emu oil and specific essential oils that acts as a safe and natural alternative to hair growth drugs such as Minoxidil, Finasteride, antiandrogens and steroids.

This detailed description will show that fully-refined Emu Oil can be used as a successful transdermal carrier of Rosemary, Thyme, Lavender and Cedarwood essential oils, oils that are proven to stimulate healthy hair growth. Together, the mixture of emu oil and specific essential oils acts as a human hair root stimulator that promotes healthy hair growth.

### Essential Oils Study

The specific combination of Rosemary, Thyme, Lavender and Cedarwood Essential Oils has been scientifically proven to grow hair in patients suffering from alopecia areata (patchy hair loss).

The results of a 1996 study published by Scottish researchers (Department of Dermatology, Aberdeen Royal Infirmary, Foresterhill, Aberdeen, Scotland) appears in the Archives of Dermatology (1998; 134:1349-1352.) The study's objective was “to investigate the efficacy of aromatherapy in the treatment of patients with alopecia areata.” (Hay)

The study consisted of a double-blind controlled trial of 86 patients for 7 months. The patients were split into two groups: an “active group” who massaged essential oils (thyme, rosemary, lavender, and cedarwood) in a mixture of carrier oils (jojoba and grapeseed) into their scalp daily; and a “control group” who massaged only carrier oils (jojoba and grapeseed) into their scalps daily.

According to the published report, “Treatment success was evaluated on sequential photographs by two dermatologists (I.C.H. and A.D.O.) independently. Similarly, the degree of improvement was measured by 2 methods: a 6-point scale and computerized analysis of traced areas of alopecia.” (Hay)

Results showed that 19 of 43 patients (44%) in the active group showed “significant” improvement in hair growth compared with just 6 of 41 patients (15%) in the control group.

The study concluded: “The results show aromatherapy to be a safe and effective treatment for alopecia areata. Treatment with these essential oils was significantly more effective than treatment with the carrier oil alone.” (Hay)

Due to their low molecular weight, essential oils are absorbed through the skin and hair follicles. Absorption can take between 15 minutes and 12 hours. However, most essential oils must be diluted in a carrier oil before being topically applied to the skin in order to prevent skin irritation. The carrier oil used to dilute the essential oils directly affects the absorption rate of the mixture, since some carrier oils are heavier than others. (Cooksley p. 15)

One carrier oil proven to effectively penetrate human skin and carry medicinal substances such as essential oils into the blood stream is Emu Oil.

### Emu Oil as a Transdermal Carrier Oil

Emu oil has been proven to enhance skin penetration, providing a faster and more effective method of delivering topical drugs and medications to the blood stream (Schatz, p. 27-28). Researchers believe emu oil is so penetrating because it is almost 100% triglyceride lipid, containing no phosphor-lipids. (Schatz p. 37)

According to one researcher, “Our skin is phosphor-lipid deficient. In other words, there's no phosphorus in our skin. If you put anything on your skin that has phosphorus in it, your skin is ‘programmed’ to keep it from penetrating. Anytime you put anything on your skin that is phosphor-lipid deficient, or has no phosphorus, it penetrates right through.” (Schatz, p. 23)

Another researcher, Dr. Margaret C. Craig-Schmidt (Associate Professor in the Department of Nutrition and Food Science, Auburn University) says emu oil's high oleic acid content may be the reason for its skin penetrating ability.

“Oleic acid is, and has been used to carry bio-active compounds through the skin, and this was the highest fatty acid that was found in emu oil. In other words, the high presence of oleic acid in the emu oil may be one of the reasons that it is able to penetrate the skin to a large degree and at the same time be able to carry active components through the skin where they can have an effect on the body.” (Schatz, p. 88)

### Emu Oil Skin Penetration Studies

Dr. G. R. Hobday of Australia published his findings on emu oil and skin penetration in his paper Emu Oil—A Clinical Appraisal of This Natural and Long Used Product. “From clinical experience with emu oil, it became obvious that its two major actions were its anti-inflammatory properties and its ability to penetrate the skin.” (Schatz, p. 34)
Dr. Paul Smith, Professor of Pathobiology, Auburn University, Alabama, conducted studies using emu oil as a transdermal carrier of medicine on mice. “In each treatment we simply took each compound and a small syringe and drew up 0.2 mils of the compound treating each mouse over that area of skin . . . . Although we did not attempt to rub the compound in, we made sure the compound covered the entire area and returned the mouse back in the cage to be left for half an hour.” (Schatz, p. 50). Blood samples were then taken from the mice and examined using a high pressure liquid chromatograph. “According to the results of two of the mice in each group, the amount of drug detected in the mouse serum was surprising,” according to Smith. (Schatz, p. 51)
Another three penetration studies were conducted by Emu Marketing Unlimited and Dr. Peter T. Pugliese, a dermatologist and biomedical consultant in Pennsylvania. The conclusion was that “Emu Oil is . . . a penetration enhancer” of medicinal substances, such as methyl nicotinate as used in their study. (Schatz, p. 61)
Emu oil is also proving to have some of the same transdermal qualities as Liposomes. Discovered in 1961, these hollow microscopic spheres are formed of thin lipid membranes used to encapsulate a drug and transport it through the bloodstream. (Schatz, p. 84) According to Dr. Alex Zemtsov (certified dermatologist and Assoc. Professor of Biochemistry and Molecular Biology at Indiana University School of Medicine & adjunct Assoc. Professor of Medical Education at Ball State), the most interesting property of emu oil is its penetrating properties. “What I mean by penetrating is that it goes through the skin barrier and directly into the skin, which is very important both for cosmetic and pharmaceutical interests, because it can go through the skin barrier and carry active ingredients into the skin.” (Schatz, p. 86)

### Emu Oil's Ability to Penetrate the Scalp and Reach the Hair Folicle

In addition to studies on emu oil's ability to penetrate the skin, successful studies have shown emu oil's specific ability to penetrate the scalp and reach the hair follicles below. Dr. Michael Holick (Professor of Medicine, Physiology, and Dermatology, Boston University School of Medicine) received U.S. Pat. No. 5,744,128 in 1998 for the topical “Use of emu oil for stimulating skin and hair growth.”

According to the patent abstract, “ . . . emu oil is useful to treat . . . disorders relating to disturbances in hair cycling such as alopecia, male pattern baldness, female baldness, and chemotherapy-induced alopecia.”

Holick published his findings in a 1996 article, “Study on Skin Enhancement and Hair Growth” in Drug and Cosmetic Industry Magazine.

“We found that there was about a 20% increase in the proliferative activity, or the growth activity of the skin in the animals . . . . And when we looked at the hair follicles, and the thickness of the skin, it showed that the hair follicles were much more robust, and that the skin thickness was remarkably increased, suggesting . . . the stimulation of skin growth and hair growth in these animals. Also, we discovered in the same test that over 80% of hair follicles that had been asleep were woken up, and began growing hair.

“A hair follicle goes through a cycle. It goes from a resting stage into an active growth period, and then it goes back to sleep again. We woke up all the hair follicles by stimulating them, and then we wanted to see if we could further stimulate these hair follicles by topically applying emu oil. We found that there was an enhancement in the growth activity of the hair follicles. So it gives us a very good scientific indication that we were stimulating skin growth.”

### CONCLUSION

By combining the skin-penetrating properties of emu oil and the hair growth-stimulating properties of essential oils of Rosemary, Thyme, Lavender and Cedarwood, it is possible to successfully promote healthy hair growth in humans where the oil treatment is massaged into the scalp once a day for at least three months and is allowed to penetrate the scalp for up to 12 hours.

What is claimed is:

1. A method for stimulating the hair root follicles on a human head in order to promote hair growth comprising the steps of: Administering emu oil combined with essential oils of Rosemary, Thyme, Lavender & Cedarwood to the scalp of the individual seeking to stimulate hair root follicles below the scalp and promote hair growth.

2. The method of claim 1, wherein the emu oil is fully refined.

3. The method of claim 1, wherein the essential oils comprise distilled plant components of Rosemary (Rosmarinus officinalis), Thyme (Thymus vulgaris), Lavender (Lavendula augustifolia), and Cedarwood (Cedrus atlanticus).

4. The method of claim 1, wherein the individual is a human male or female.

5. The method of claim 1, wherein the preparation is administered topically to an area of the scalp where hair growth promotion is desired.

6. The method of claim 1, wherein the preparation is massaged into the scalp.

7. The method of claim 1, wherein the preparation is administered at least daily for a period of at least 3 months.
